SEQUENCE LISTING (4 sequences)

SEQ ID NO: 1 - protein, 332 aa, Oryctolagus cuniculus
MAALKDQLIHNLLKEEHVPQNKITVVGVGAVGMACAISILMKDLADELALVDVMEDKLKGEMMDLQHGSLFLRTPKIVSGKDYSVTANSKLVIITAGARQQEGESRLNLVQRNVNIFKFIIPNVVKYSPHCKLLVVSNPVDILTYVAWKISGFPKNRVIGSGCNLDSARFRYLMGERLGVHALSCHGWILGEHGDSSVPVWSGMNVAGVSLKTLHPELGTDADKEQWKQVHKQVVDSAYEVIKLKGYTSWAIGLSVADLAESIMKNLRRVHPISTMIKGLYGIKEDVFLSVPCVLGQNGISDVVKVTLTSEEEAHLKKSADTLWGIQKELQF

SEQ ID NO: 2 - protein, 334 aa, Homo sapiens
MATLKEKLIAPVAEEEATVPNNKITVVGVGQVGMACAISILGKSLADELALVDVLEDKLKGEMMDLQHGSLFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 3 - protein, 332 aa, Homo sapiens
MATLKDQLIYNLLKEEQTPQNKITVVGVGAVGMACAISILMKDLADELALVDVIEDKLKGEMMDLQHGSLFLRTPKIVSGKDYNVTANSKLVIITAGARQQEGESRLNLVQRNVNIFKFIIPNVVKYSPNCKLLIVSNPVDILTYVAWKISGFPKNRVIGSGCNLDSARFRYLMGERLGVHPLSCHGWVLGEHGDSSVPVWSGMNVAGVSLKTLHPDLGTDKDKEQWKEVHKQVVESAYEVIKLKGYTSWAIGLSVADLAESIMKNLRRVHPVSTMIKGLYGIKDDVFLSVPCILGQNGISDLVKVTLTSEEEARLKKSADTLWGIQKELQF

SEQ ID NO: 4 - protein, 334 aa, Sus scrofa domesticus
MATLKEKLIAPVAEEETTIPNNKITVVGVGQVGMACAISILGKSLTDELALVDVLEDKLKGEMMDLQHGSLFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGVHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVQGMYGIENEVFLSLPCVLNARGLTSVINQKLKDDEVAQLKNSADTLWGIQKDLKDL

(19) United States
(12) Patent Application Publication
(10) Pub. No.: US 2023/0348520 A1
(43) Pub. Date: Nov. 2, 2023

(54) COMPOUND COMPRISING BETA-NICOTINAMIDE MONONUCLEOTIDE OR PHARMACOLOGICALLY ACCEPTABLE SALT THEREOF, AND METHOD FOR EVALUATING QUALITY AND METHOD FOR ASSESSING ENZYMATIC REACTIVITY OF SAID COMPOUND

(71) Applicant: ORIENTAL YEAST CO., LTD., Tokyo (JP)

(72) Inventors: Yukiko TAKETANI, Shiga (JP); Hirokazu MATSUKAWA, Shiga (JP); Yoshiko SUNAHARA, Shiga (JP)

(21) Appl. No.: 17/907,346

(22) PCT Filed: Feb. 10, 2021
(86) PCT No.: PCT/JP2021/004912

§ 371 Date: Sep. 26, 2022

(30) Foreign Application Priority Data
Mar. 27, 2020 (JP) 2020-058429

(51) Int. Cl. C07H 19/048; C12Q 1/32; B01D 15/08

(52) U.S. Cl. CPC C07H 19/048; C12Q 1/32; B01D 15/08; C12Y 101/01027

(57) ABSTRACT

A compound includes β-nicotinamide mononucleotide or a pharmacologically acceptable salt thereof. A purity of the compound as measured through HPLC is 95% or higher. A reactivity of the compound with lactate dehydrogenase is 30 units or higher.

### TECHNICAL FIELD

The present invention relates to a compound including β-nicotinamide mononucleotide or a pharmacologically acceptable salt thereof, and a method of evaluating a quality of the compound and a method of determining an enzymatic reactivity of the compound.

### BACKGROUND ART

β-Nicotinamide mononucleotide (hereinafter may be referred to as “β-NMN”) is an intermediate metabolite in biosynthesis of coenzyme NAD⁺. In recent years, it is reported, for example, that β-NMN has the effect of enhancing the ability to secrete insulin in aged mice and the effect of drastically enhancing insulin sensitivity and secretion in model mice of Type 2 diabetes caused by high-fat diets and aging (see, for example, PTL 1), β-NMN is involved with control of a circadian rhythm (see, for example, PTL 2), and β-NMN has the effect of remarkably enhancing the mitochondrial functions in aged muscles. In addition, it is also reported that administration of β-NMN is useful for improvement and prevention of symptoms of various age-related diseases due to obesity, increase in the blood lipid level, decrease in insulin sensitivity, decrease in memory, and degradation in the functions of eyes such as macular degeneration (see, for example, PTL 3). Moreover, administration of β-NMN is expected to produce the anti-aging effect caused by increasing the NAD⁺ amount in living bodies to activate a Sirtuin gene, thereby preventing and delaying age-related decrease in physical functions of living bodies (see, for example, PTL 4).

Meanwhile, when β-NMN is applied to, for example, pharmaceuticals, supplements, and cosmetics, there are some proposals to increase the purity of β-NMN and crystallize β-NMN for improving storage stability (see, for example, PTLs 5 and 6).

### CITATION LIST

### Patent Literature

PTL 1: U.S. Pat. No. 7,737,158

PTL 2: US Patent Application Publication No. 2011/123510

PTL 3: International Publication No. WO2014/146044

PTL 4: International Publication No. WO2017/200050

PTL 5: Japanese Patent Application Laid-Open No. 2018-534265

PTL 6: International Publication No. WO2018/047715

### SUMMARY OF INVENTION

### Technical Problem

The present inventors have obtained the following findings. Specifically, although β-nicotinamide mononucleotide that the present inventors produced by themselves and β-NMN products available on the market similarly had high purities as measured through high performance liquid chromatography (hereinafter may be referred to as “HPLC”) did not produce similar effects when administered to living bodies even at the same doses. In addition, even when crystallized for increasing purities, they did not produce similar effects when administered to living bodies even at the same doses. In other words, some β-NMNs have different physiological activities even when they have comparable purities as measured through HPLC. That is why there is a need to provide β-NMN having a higher physiological activity.

The present invention meets such a demand and overcomes the current circumstances, aiming to solve the above existing problems and achieve the following object. Specifically, an object of the present invention is to provide: a compound including β-nicotinamide mononucleotide that has a high purity as measured through HPLC and exhibits a high enzymatic reactivity to have a high physiological activity; a compound including a pharmacologically acceptable salt of the β-nicotinamide mononucleotide; a method of evaluating a quality of a compound including β-nicotinamide mononucleotide or a pharmacologically acceptable salt thereof, and a method of determining an enzymatic reactivity of the compound.

### Solution to Problem

The present inventors conducted intensive studies in order to achieve the above object, and have found that the reason why β-NMN products exhibit different physiological activities even when the purities thereof as measured through HPLC are comparable is because β-NMNs in these products have different enzymatic reactivities. On the basis of this finding, the present inventors have completed the present invention.

The present invention is based on the above finding obtained by the present inventors, and means for solving the above problems are as follows.

<1> A compound, including:

β-nicotinamide mononucleotide or a pharmacologically acceptable salt thereof,

wherein a purity of the compound as measured through HPLC is 95% or higher, and a reactivity of the compound with lactate dehydrogenase is 30 units or higher.

<2> The compound according to <1> above, wherein the purity of the compound as measured through HPLC is 98% or higher, and the reactivity of the compound with lactate dehydrogenase is 33 units or higher.

<3> The compound according to <1> or <2> above, wherein the compound is in crystalline form.

<4> The compound according to any one of <1> to <3> above, wherein the lactate dehydrogenase is lactate dehydrogenase derived from mammalian skeletal muscle.

<5> The compound according to <4> above, wherein the lactate dehydrogenase includes an amino acid sequence of SEQ ID NO: 1.

<6> The compound according to any one of <1> to <5> above, wherein the compound is substantially free from nicotinamide dinucleotide.

<7> A method of evaluating a quality of a compound including β-nicotinamide mononucleotide or a pharmacologically acceptable salt thereof, the method including:
evaluating the quality of the compound including the β-nicotinamide mononucleotide or the pharmacologically acceptable salt thereof based on an indication that a purity of the compound as measured through HPLC is 95% or higher, and a reactivity of the compound with lactate dehydrogenase is 30 units or higher.

<8> A method of determining an enzymatic reactivity of a compound including β-nicotinamide mononucleotide or a pharmacologically acceptable salt thereof, the method including:
determining a reactivity, with lactate dehydrogenase, of the compound including the β-nicotinamide mononucleotide or the pharmacologically acceptable salt thereof based on an indication that a purity of the compound as measured through HPLC is 95% or higher, and the reactivity of the compound with the lactate dehydrogenase is 30 units or higher.

### Advantageous Effects of Invention

According to the present invention, it is possible to solve the above existing problems to achieve the above object and provide: a compound including β-nicotinamide mononucleotide that has a high purity as measured through HPLC and exhibits a high enzymatic reactivity to have a high physiological activity; a compound including a pharmacologically acceptable salt of the β-nicotinamide mononucleotide; a method of evaluating a quality of a compound including β-nicotinamide mononucleotide or a pharmacologically acceptable salt thereof; and a method of determining an enzymatic reactivity of the compound.

### BRIEF DESCRIPTION OF DRAWINGS

FIG. 1 is a view illustrating a reaction between β-NMN and lactate dehydrogenase.

FIG. 2 is a graph illustrating enzymatic reactivities, with lactate dehydrogenase (SEQ ID NO: 1), of a β-NMN compound as one example of the present invention and commercially available β-NMN products, as measured in Test Example 1.

FIG. 3A is a graph illustrating enzymatic reactivities, with porcine lactate dehydrogenase (LDH) 1 (SEQ ID NO: 4), of a β-NMN compound as one example of the present invention and commercially available β-NMN products, as measured in Test Example 3.

FIG. 3B is a graph illustrating enzymatic reactivities, with human lactate dehydrogenase (LDH) 1 (SEQ ID NO: 2), of a β-NMN compound as one example of the present invention and commercially available β-NMN products, as measured in Test Example 3.

FIG. 3C is a graph illustrating enzymatic reactivities, with human lactate dehydrogenase (LDH) 5 (SEQ ID NO: 3), of a β-NMN compound as one example of the present invention and commercially available β-NMN products, as measured in Test Example 3.

FIG. 4A is a graph illustrating relative activity values of lactate dehydrogenase (SEQ ID NO: 1) to a β-NMN compound as one example of the present invention and a commercially available β-NMN product 1 at various concentrations, as measured in Test Example 4.

FIG. 4B is a graph illustrating relative activity values of lactate dehydrogenase (SEQ ID NO: 1) to a β-NMN compound as one example of the present invention and a commercially available β-NMN product 2 at various concentrations, as measured in Test Example 4.

FIG. 4C is a graph illustrating relative activity values of lactate dehydrogenase (SEQ ID NO: 1) to a β-NMN compound as one example of the present invention and a commercially available β-NMN product 3 at various concentrations, as measured in Test Example 4.

FIG. 4D is a graph illustrating relative activity values of human lactate dehydrogenase (LDH) 1 (SEQ ID NO: 2) to a β-NMN compound as one example of the present invention and a commercially available β-NMN product 1 at various concentrations, as measured in Test Example 4.

FIG. 4E is a graph illustrating relative activity values of human lactate dehydrogenase (LDH) 1 (SEQ ID NO: 2) to a β-NMN compound as one example of the present invention and a commercially available β-NMN product 2 at various concentrations, as measured in Test Example 4.

FIG. 4F is a graph illustrating relative activity values of human lactate dehydrogenase (LDH) 1 (SEQ ID NO: 2) to a β-NMN compound as one example of the present invention and a commercially available β-NMN product 3 at various concentrations, as measured in Test Example 4.

FIG. 5A is a graph illustrating cytotoxicity rates determined based on intra- and extracellular AST activities when a β-NMN compound as one example of the present invention is used, as measured in Test Example 5.

FIG. 5B is a graph illustrating cytotoxicity rates determined based on intra- and extracellular AST activities when a β-NMN product 3 is used, as measured in Test Example 5.

FIG. 6A is a graph illustrating an intracellular NAD content when a β-NMN compound as one example of the present invention is used, as measured in Test Example 5.

FIG. 6B is a graph illustrating an intracellular NAD content when a β-NMN product 3 is used, as measured in Test Example 5.

### DESCRIPTION OF EMBODIMENTS

### (Compound)

The compound of the present invention is a compound including β-nicotinamide mononucleotide or a pharmacologically acceptable salt thereof. A purity of the compound as measured through HPLC is 95% or higher. A reactivity of the compound with lactate dehydrogenase is 30 units or higher.

The β-nicotinamide mononucleotide has two kinds, α and β, as optical isomers. The structure of the β-nicotinamide mononucleotide according to the present invention (CAS NO: 1094-61-7) is as follows.

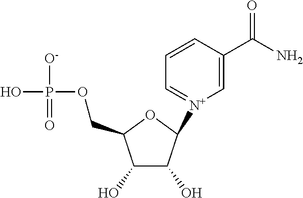 [SMILES: NC(=O)c1ccc[n+]([C@@H]2O[C@H](COP(=O)([O-])O)[C@@H](O)[C@H]2O)c1]

The compound including the β-NMN or pharmacologically acceptable salt thereof according to the present invention (hereinafter may be referred to as a “β-NMN compound”) may be prepared by any method. For example, the β-NMN that is artificially synthesized by, for example, a chemical synthesis method, an enzymatic method, or a fermentation method, followed by purification can be used as an active ingredient. Alternatively, the β-NMN, which is an ingredient that is ubiquitously present in living bodies, can be obtained through extraction and/or purification from natural materials such as animals, plants, and microorganisms, and the obtained β-NMN can be used as an active ingredient. Still alternatively, purified β-NMN that is commercially available may be used.

Examples of the chemical synthesis method of synthesizing the β-NMN include allowing nicotinamide and L-ribose tetraacetate to react and phosphorylating the obtained nicotinamide mononucleoside to produce the β-NMN. Also, examples of the enzymatic method include: producing the β-NMN from nicotinamide and 5′-phosphoribosyl-1′-pyrophosphoric acid (hereinafter may be referred to as “PRPP”) by the action of nicotinamide phosphoribosyl transferase (hereinafter may be referred to as “NAMPT”); and producing the β-NMN from nicotinamide riboside by the action of nicotinamide riboside kinase. Also, examples of the fermentation method include producing the β-NMN from nicotinamide using the metabolic system of a microorganism that is expressing NAMPT.

The β-NMN may be a pharmacologically acceptable salt thereof. The pharmacologically acceptable salt of the β-NMN may be an inorganic acid salt or may be an organic acid salt having a basic site such as an amine. Examples of the acid that forms such an acid salt include acetic acid, benzenesulfonic acid, benzoic acid, camphorsulfonic acid, citric acid, ethenesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, and p-toluenesulfonic acid. Also, the pharmacologically acceptable salt of the β-NMN may be an alkali salt or may be an organic salt having an acidic site such as carboxylic acid. Examples of the base that forms such an acid salt include those derived from alkali metal salts or alkaline earth metal salts, such as sodium hydride, potassium hydroxide, calcium hydroxide, aluminum hydroxide, lithium hydroxide, and magnesium hydroxide; and zinc hydroxide, ammonia, trimethyl ammonia, triethyl ammonia, ethylene diamine, lysine, arginine, ornithine, choline, N,N′-dibenzylethylenediamine, chloroprocaine, procaine, diethanolamine, N-benzylphenethylamine, diethylamine, piperazine, tris(hydroxymethyl)-aminomethane, and tetramethylammonium hydroxide.

The β-NMN compound according to the present invention may be in crystalline form or may be amorphous (non-crystalline) form. However, in order to reduce impurities to a lower level and more increase stability as the compound, preferable is a crystallized β-NMN compound obtained through crystallization using a methanol solution or a solution containing an alcohol such as ethanol. A crystallization method of the β-NMN compound is not particularly limited and may be appropriately selected from known methods. The crystallization method can be performed according to, for example, the method described in Japanese Patent Application Laid-Open No. 2018-534265 or International Publication No. WO2018/047715.

### <HPLC Purity>

The purity of the β-NMN compound according to the present invention is not particularly limited and may be appropriately selected in accordance with the intended purpose, as long as the purity thereof as measured through HPLC (hereinafter may be referred to as an “HPLC purity”) is 95% or higher. The HPLC purity is preferably 98% or higher.

The HPLC purity in the present invention refers to a ratio of an NMN-derived peak area to the total of various peak areas detected when a sample containing β-NMN is measured through HPLC. Specifically, the HPLC purity can be calculated from the following formula.

HPLC purity (%)=(β-NMN-derived peak area)/(total of peak areas measured)×100 —Formula—

In the present invention, an HPLC analysis method used when measuring the HPLC purity is not particularly limited and may be appropriately selected in accordance with the intended purpose, as long as it is a method or condition that can efficiently separate and measure the β-NMN. For example, the HPLC purity can be measured by the method described in “Yoshino, et al., Cell Metabolism, 2011, vol. 14, pp. 528-536.” using Hypercarb™ (length: 15 cm, inner diameter: 4.6 mm, particle diameter: 3 μm, obtained from Thermo Fisher Scientific) as a column or by the method described in “Journal of Vitamins, 1990, Vol. 64, No. 1, pp. 19 to 25” using a TSK-GEL ODS column (length: 15 cm, inner diameter: 4.6 mm, particle diameter: 5 μm, obtained from TOSOH CORPORATION) as a column. In the present invention, the HPLC purity was measured by the following method.

As an HPLC apparatus, HPLC System Prominence (obtained from SHIMADZU CORPORATION) is used.
A β-NMN sample is dissolved in distilled water so as to have a concentration of 2 mM, and the resulting solution is used as a sample liquid.
10 μL of the sample liquid is applied to a TSK-GEL ODS-80TS column (length: 15 cm, inner diameter: 4.6 mm, particle diameter: 5 μm, obtained from TOSOH CORPORATION).
A β-NMN fraction adsorbed to the column is separated by the following method.

Using 50 mM Tris-acetic acid (pH 7.5)/methanol as an eluent, the β-NMN is eluted and separated at a methanol concentration gradient of from 0 to 15% and an adjusted flow rate of 0.7 mL/min, followed by measurement of absorbance at 260 nm.

### <Enzymatic Reactivity>

Also, the reactivity of the β-NMN compound according to the present invention with lactate dehydrogenase (hereinafter may be referred to as “enzymatic reactivity”) is not particularly limited and may be appropriately selected, as long as the enzymatic reactivity is 30 units or higher. The enzymatic reactivity is preferably 33 units or higher.

The lactate dehydrogenase in the present invention (hereinafter may be referred to as “LDH”) is a dehydrogenase that is also referred to as L-lactate dehydrogenase (EC 1.1.1.27).

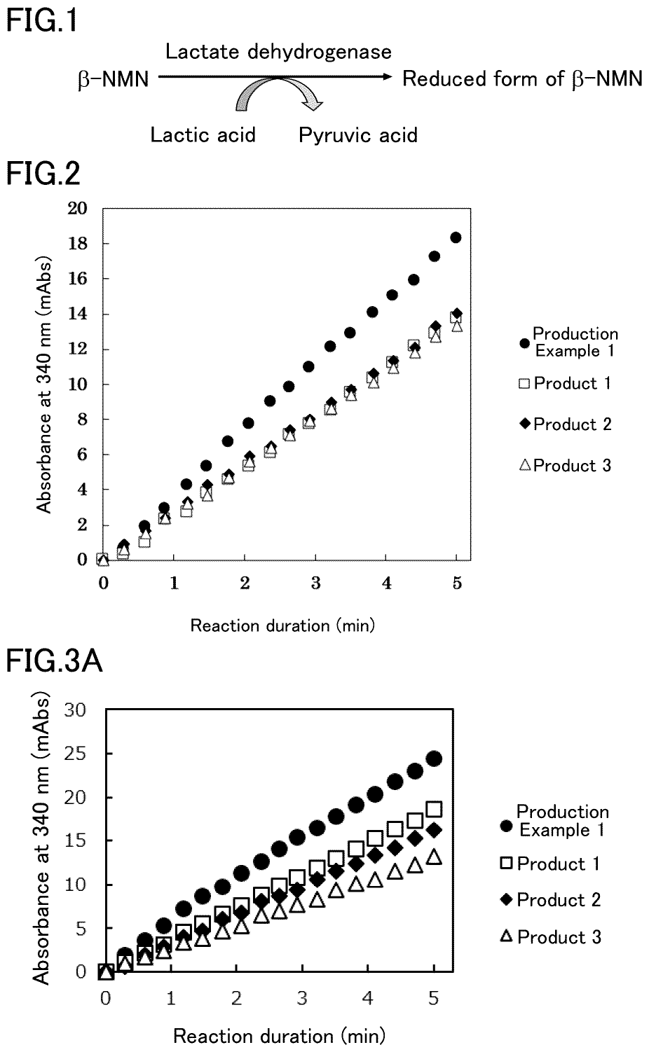

The lactate dehydrogenase is preferably lactate dehydrogenase derived from mammalian skeletal muscle, and more preferably lactate dehydrogenase (LDH) 5 (hereinafter may be referred to as “R-LDHS”). The R-LDHS has an amino acid sequence as set forth in SEQ ID NO: 1, is composed of a tetramer of sub-units, and is derived from skeletal muscle. Other lactate dehydrogenases of any mammal can also be used, such as porcine lactate dehydrogenase (LDH) 1, human lactate dehydrogenase (LDH) 1, and human lactate dehydrogenase (LDH) 5. The LDH catalyzes dehydrogenation of lactic acid as a substrate, to convert the lactic acid to pyruvic acid. At this time, the β-NMN is converted to a reduced form of β-NMN (see FIG. 1).

In the present invention, the enzymatic reactivity refers to a value obtained through measurement using the following reagents and method.

### <R1 Reagent>

### 80 mM Tris-HCl (pH 8.5)

### 19 mM β-NMN

(The purity of the β-NMN in a measurement sample is assumed to be 100%. Also, the β-NMN is dissolved in an 80 mM sodium carbonate solution in advance.)

### <R2 Reagent>

### 100 mM Tris-HCl (pH 8.5)

100 mM L-lactic acid

### <Enzyme Solution>

The enzyme solution is prepared so that the LDH is to be 245 U/mL as a final concentration in a reaction liquid.

In the present invention, the activity unit (U) of the LDH is an activity unit measured by the human LDH measurement method according to IFCC when using NAD as a coenzyme.

### <Measurement of Activity>

An enzyme activity is measured using a 7180 type Hitachi automatic analyzer (obtained from Hitachi High-Tech Corporation). Measurement parameters are as follows.

### —Measurement Parameters—

Analysis method: Rate A
Measurement wavelength (sub/main): 405 nm/340 nm
Reaction duration: 10 minutes
Photometric points: 20 to 24
Sample liquid (enzyme solution): 18 μL
R1 reagent: 120 μL
R2 reagent: 87 μL

### —Measurement Procedure—

18 μL of the enzyme solution and 120 μL of the R1 reagent are mixed. The resulting mixture is incubated at 37° C. for 4.5 minutes (photometric points 1 to 16), followed by addition of 87 μL of the R2 reagent, to initiate reaction (photometric point 17). From the absorbance of the measurement sample one to two minutes after initiation of the reaction (photometric points 20 to 24), the absorbance of water (blank) at the same photometric points is subtracted, to calculate a value of change in absorbance per minute (ΔmAbs/min). Regarding the activity unit (the unit of reactivity with the lactate dehydrogenase R-LDH5), 0.1 mAbs as the value of change in absorbance per minute is defined as one unit.

Preferably, the β-NMN compound according to the present invention is substantially free from nicotinamide dinucleotide (hereinafter may be referred to as “NAD”). This is because the β-NMN compound containing the NAD cannot be accurately measured for reactivity with lactate dehydrogenase.

In the present invention, being substantially free from nicotinamide dinucleotide means that the NAD in the β-NMN compound is not detected through the above HPLC analysis. Note that, this should not be construed as excluding embodiments in which the NAD is contained in preparations or foods and drinks in which the β-NMN compound according to the present invention is to be included.

The β-NMN compound of the present invention has not only a high purity as measured through HPLC but also a high enzymatic reactivity with lactate dehydrogenase. Therefore, as compared with the traditional β-NMN products (drug substances), the β-NMN compound of the present invention has high bioavailability. The β-NMN compound has both a high quality and a high physiological action as β-nicotinamide mononucleotide or a pharmacologically acceptable salt thereof and a product containing it. Thus, higher effects can be obtained as not only pharmaceuticals containing the β-NMN as an active ingredient but also foods and drinks (which include, but are not limited to, supplements) and raw materials of feeds. Alternatively, administering or intaking these can produce higher effects.

### (Method of Evaluating Quality of Compound)

The present inventive method of evaluating a quality of a compound is a method of evaluating a quality of the compound including β-nicotinamide mononucleotide or a pharmacologically acceptable salt thereof. The method includes an evaluation step; and if necessary, further includes other steps such as a measurement step.

### <Evaluation Step>

The evaluation step in the present inventive method of evaluating a quality of a compound is evaluating a quality of a compound including β-nicotinamide mononucleotide or a pharmacologically acceptable salt thereof based on an indication that a purity of the compound as measured through HPLC is 95% or higher, and a reactivity of the compound with lactate dehydrogenase is 30 units or higher.

The indication of the purity as measured through HPLC is not particularly limited and may be appropriately selected in accordance with the intended purpose, as long as the purity is 95% or higher. The purity is preferably 98% or higher.

The indication of the reactivity with lactate dehydrogenase is not particularly limited and may be appropriately selected in accordance with the intended purpose, as long as the reactivity is 30 units or higher. The reactivity is preferably 33 units or higher.

### —Evaluation—

In the evaluation step in the present inventive method of evaluating the quality of the compound, the quality of an evaluation target—the compound including β-nicotinamide mononucleotide or a pharmacologically acceptable salt thereof—is determined to be good when the purity as measured through HPLC is 95% or higher and the reactivity with lactate dehydrogenase is 30 units or higher. More specifically, when the purity as measured through HPLC is 95% or higher and the reactivity with lactate dehydrogenase is 30 units or higher, the evaluation target is evaluated as a compound including β-nicotinamide mononucleotide or a pharmacologically acceptable salt thereof having high bioavailability.

The purity as measured through HPLC and the reactivity with lactate dehydrogenase may be measured during the implementation of the present inventive method of evaluating a quality of the compound. Alternatively, the purity as measured through HPLC and the reactivity with lactate dehydrogenase may be measured separately from the implementation of the method of the present invention.

### <Other Steps>

The other steps in the present inventive method of evaluating a quality of the compound are not particularly limited and may be appropriately selected in accordance with the intended purpose, as long as the other steps do not impair the effects of the present invention. Examples the other steps include a measurement step.

### —Measurement Step—

The measurement step in the present inventive method of evaluating a quality of the compound is measuring the evaluation target—the compound including β-nicotinamide mononucleotide or a pharmacologically acceptable salt thereof—for the purity as measured through HPLC and the reactivity with lactate dehydrogenase.

The purity as measured through HPLC and the reactivity with lactate dehydrogenase can be measured in the same manner as in the section <HPLC purity> and the section <Enzymatic reactivity> in the (Compound) described above.

### (Method of Determining Enzymatic Reactivity of Compound)

The present inventive method of determining an enzymatic reactivity of a compound is a method of determining an enzymatic reactivity of the compound including β-nicotinamide mononucleotide or a pharmacologically acceptable salt thereof. The method includes a determination step; and if necessary, further includes other steps.

### <Determination Step>

The determination step in the present inventive method of determining an enzymatic reactivity of a compound is determining an enzymatic reactivity of a compound including β-nicotinamide mononucleotide or a pharmacologically acceptable salt thereof based on an indication that a purity of the compound as measured through HPLC is 95% or higher, and a reactivity of the compound with lactate dehydrogenase is 30 units or higher.

The indication of the purity as measured through HPLC is not particularly limited and may be appropriately selected in accordance with the intended purpose, as long as the purity is 95% or higher. The purity is preferably 98% or higher.

The indication of the reactivity with lactate dehydrogenase is not particularly limited and may be appropriately selected in accordance with the intended purpose, as long as the reactivity is 30 units or higher. The reactivity is preferably 33 units or higher.

### —Determination—

In the determination step in the present inventive method of determining the enzymatic reactivity of the compound, the enzymatic reactivity of a determination target—the compound including β-nicotinamide mononucleotide or a pharmacologically acceptable salt thereof—is determined to be high when the purity as measured through HPLC is 95% or higher and the reactivity with lactate dehydrogenase is 30 units or higher. More specifically, when the purity as measured through HPLC is 95% or higher and the reactivity with lactate dehydrogenase is 30 units or higher, the evaluation target is determined as a compound including β-nicotinamide mononucleotide or a pharmacologically acceptable salt thereof having a high reactivity with lactate dehydrogenase.

The purity as measured through HPLC and the reactivity with lactate dehydrogenase may be measured during the implementation of the present inventive method of determining an enzymatic reactivity of the compound. Alternatively, the purity as measured through HPLC and the reactivity with lactate dehydrogenase may be measured separately from the implementation of the method of the present invention.

### <Other Steps>

The other steps in the present inventive method of determining an enzymatic reactivity of the compound are not particularly limited and may be appropriately selected in accordance with the intended purpose, as long as the other steps do not impair the effects of the present invention. Examples of the other steps include a measurement step.

### —Measurement Step—

The measurement step in the present inventive method of determining an enzymatic reactivity of the compound is measuring the evaluation target—the compound including β-nicotinamide mononucleotide or a pharmacologically acceptable salt thereof—for the purity as measured through HPLC and the reactivity with lactate dehydrogenase.

The purity as measured through HPLC and the reactivity with lactate dehydrogenase can be measured in the same manner as in the section <HPLC purity> and the section <Enzymatic reactivity> in the (Compound) described above.

According to the present inventive method of evaluating the compound or determining an enzymatic reactivity of the compound, it is possible to evaluate or determine bioavailability of β-nicotinamide mononucleotide or a pharmacologically acceptable salt thereof, which cannot be evaluated based only on the HPLC purity thereof. This can provide a compound including β-NMN or a pharmacologically acceptable salt thereof having a high quality.

Also, the present invention encompasses a method of producing a compound including β-NMN or a pharmacologically acceptable salt thereof having a high quality, where the method encompasses the method of evaluating the compound or determining an enzymatic reactivity of the compound.

### EXAMPLES

The present invention will be described below by way of Production Examples and Test Examples. However, the present invention should not be construed as being limited to these Production Examples and Test Examples.

### Production Example 1

<Enzymatic treatment of β-NMN>

About 100 mg of a β-NMN reagent (obtained from ORIENTAL YEAST CO., LTD., amorphous) was dissolved in a 100 mM phosphate buffer (pH 6.5). The solution was adjusted to pH 6.0 with 4N KOH. After that, 10,000 U of R-LDH5 derived from skeletal muscle, followed by reaction at 10° C. for 10 minutes. The reaction liquid was ultrafiltered, and the filtrate was collected to recover β-NMN from which LDH had been removed. The recovered β-NMN-containing solution was lyophilized to produce a powdery β-NMN sample.

### <Crystallization Treatment>

5 mL of an aqueous solution prepared by mixing water and ethanol at a volume ratio of 1:2 was weighed in a test tube. The β-NMN sample was dissolved therein to prepare an aqueous saturated solution of NMN. After that, when the aqueous solution was left to stand still at 25° C. for 3 days, crystals precipitated. Subsequently, the solution containing the crystals was centrifuged to remove the supernatant. The obtained crystals were suspended in an excessive amount of ethanol, followed by centrifuging, and the supernatant was removed.

Subsequently, the resulting product was heated and dried at 60° C. for 1 hour, to prepare crystals.

### Production Examples 2 to 4

β-NMN reagents of different production lots were used. The enzymatic treatment and the crystallization treatment were performed in the same manner as in Production Example 1, to produce β-NMN compounds of the present invention.

### Test Example 1

In addition to the β-NMN compound produced in Production Example 1, the following β-NMN products available on the market were used in the present test example. All of the following β-NMN products had been subjected to a crystallization treatment.

β-NMN product 1 (a product by the fermentation method, obtained from Company A)
β-NMN product 2 (a chemically synthesized product, obtained from Company A)
β-NMN product 3 (produced by an unknown method, obtained from Company B)

### <Measurement of HPLC Purity>

By the following method, the β-NMN compound of the present invention (Production Example 1) and β-NMN products 1 to 3 were measured for the HPLC purity. The obtained results are presented in Table 1 below. Note that, the measurement results are average values in the measurement performed three times.

### [Measurement of HPLC Purity]

As an HPLC apparatus, HPLC System Prominence (obtained from SHIMADZU CORPORATION) was used.
A β-NMN sample was dissolved in distilled water so as to have a concentration of 2 mM, and the resulting solution was used as a sample liquid.
10 μL of the sample liquid was applied to a TSK-GEL ODS-80TS column (length: 15 cm, inner diameter: 4.6 mm, particle diameter: 5 μm, obtained from TOSOH CORPORATION).
A β-NMN fraction adsorbed to the column was separated by the following method.

Using 50 mM Tris-acetic acid (pH 7.5)/methanol as an eluent, the β-NMN sas eluted and separated at a methanol concentration gradient of from 0 to 15% and an adjusted flow rate of 0.7 mL/min, followed by measurement of absorbance at 260 nm.

The HPLC purity was calculated from the following formula using the total peak area and the peak area of the β-NMN in the obtained HPLC chart.

HPLC purity (%)=(β-NMN-derived peak area)/(total of peak areas measured)×100 —Formula —

### <Evaluation of Enzymatic Reactivity>

By the following method, the β-NMN compound of the present invention (Production Example 1) and β-NMN products 1 to 3 were measured for the enzymatic reactivity with R-LDHS (SEQ ID NO: 1). The obtained results are presented in Table 1 and FIG. 2. Note that, the measurement results are average values in the measurement performed three times.

### [Evaluation of Enzymatic Reactivity]

### —R1 Reagent—

### 80 mM Tris-HCl (pH 8.5)

### 19 mM β-NMN

(The purity of the β-NMN in a measurement sample was assumed to be 100%.
Also, the β-NMN was dissolved in an 80 mM sodium carbonate solution in advance.)

### —R2 Reagent—

### 100 mM Tris-HCl (pH 8.5)

100 mM L-lactic acid

### —Enzyme Solution—

The enzyme solution was prepared so that the R-LDH5 was to be 245 U/mL as a final concentration in a reaction liquid.

### —Measurement of Activity—

An enzyme activity was measured using a 7180 type Hitachi automatic analyzer (obtained from Hitachi High-Tech Corporation). Measurement parameters are as follows.

### —Measurement Parameters—

Analysis method: Rate A
Measurement wavelength (sub/main): 405 nm/340 nm
Reaction duration: 10 minutes
Photometric points: 20 to 24
Sample liquid (enzyme solution): 18 μL

### R1 Reagent: 120 μL

### R2 Reagent: 87 μL

### —Measurement Procedure—

18 μL of the enzyme solution and 120 μL of the R1 reagent were mixed. The resulting mixture was incubated at 37° C. for 4.5 minutes (photometric points 1 to 16), followed by addition of 87 μL of the R2 reagent, to initiate reaction (photometric point 17). Measurements were taken for five minutes after initiation of the reaction. From the absorbance of the measurement sample at minute one to minute two after the initiation (photometric points 20 to 24), the absorbance of water (blank) at the same photometric points was subtracted, to calculate a value of change in absorbance per minute (ΔmAbs/min). Regarding the activity unit, 0.1 mAbs as the value of change in absorbance per minute was defined as one unit.

TABLE 1

| | Production | β-NMN | β-NMN | β-NMN |
|---|---|---|---|---|
| | Example 1 | product 1 | product 2 | product 3 |
| HPLC purity (%) | 99.9 | 99.9 | 99.0 | 99.9 |
| Enzymatic | 39 | 23 | 27 | 26 |
| reactivity (unit) | | | | |

From the results of Table 1, although each of the β-NMN compound of Production Example 1 and β-NMN products 1 to 3 has a HPLC purity of 99% or higher, the enzymatic reactivity was 39 units for Production Example 1 while products 1 to 3 were low; i.e., from 23 to 26 units.

Note that, each of the β-NMN compound of Production Example 1 and β-NMN products 1 to 3 substantially did not contain NAD because any peaks attributable to NAD were not observed in HPLC.

### Test Example 2

In the same manner as in Test Example 1, the β-NMN compounds produced in Production Examples 2 to 4 were measured for the HPLC purity and the enzymatic reactivity. As a result, even when raw materials of different production lots were used, the β-NMN compounds of the present invention were confirmed to be high in both the HPLC purity and the enzymatic reactivity. These results are presented in Table 2 below in conjunction with the test results of Production Example 1. Note that, the measurement results are average values in the measurement performed three times.

Note that, each of the β-NMN compounds of Production Examples 2 to 4 substantially did not contain NAD because any peaks attributable to NAD were not observed in HPLC.

TABLE 2

| | Production | Production | Production | Production |
|---|---|---|---|---|
| | Example 1 | Example 2 | Example 3 | Example 4 |
| HPLC purity (%) | 99.9 | 99.98 | 99.8 | 99.7 |
| Enzymatic | 39 | 40 | 38 | 35 |
| reactivity (unit) | | | | |

### Test Example 3

<Evaluation of Enzymatic Reactivity with LDH Derived from Other Animal Species>

In order to confirm whether a similar tendency would be observed between LDH derived from other animal species and LDH derived from a human, the β-NMN compound produced in Production Example 1 and β-NMN products 1 to 3 the same as those used in Test Example 1 were evaluated for the enzymatic reactivity with LDHs ((A) porcine LDH1, (B) human LDH1, and (C) human LDH5).

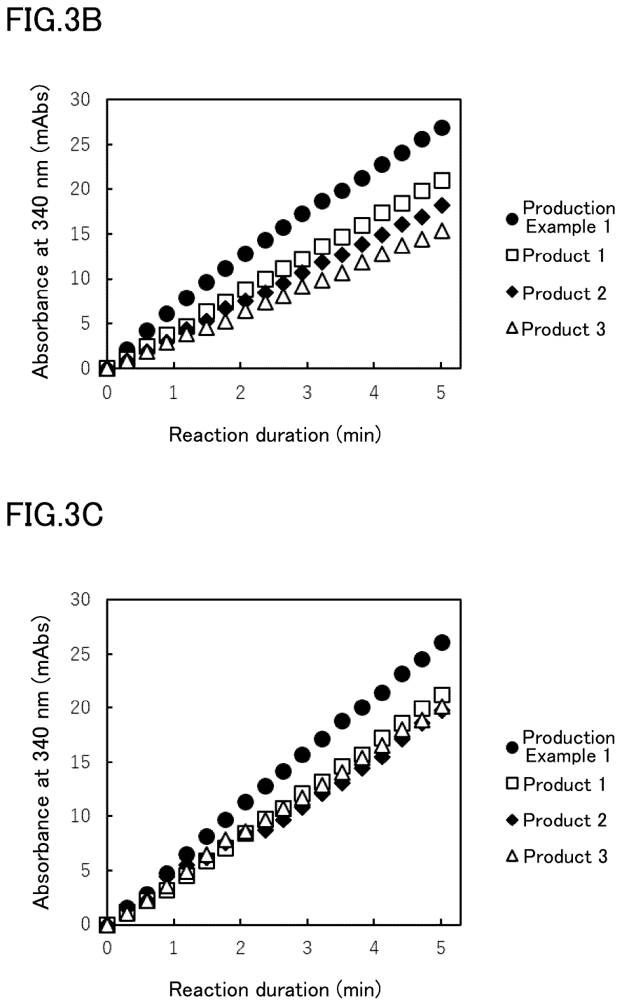

The evaluation of the enzymatic reactivity was performed in the same manner as in Test Example 1 except that the following enzyme solutions were used. The obtained results are presented in FIGS. 3A to 3C (FIG. 3A: porcine LDH1, FIG. 3B: human LDH1, and FIG. 3C: human LDH5).

### —Enzyme Solutions—

### (A) Porcine LDH1

An enzyme solution was prepared so that the LDH was to be 45 U/mL as a final concentration in a reaction liquid.

### (B) Human LDH1

An enzyme solution was prepared so that the LDH was to be 45 U/mL as a final concentration in a reaction liquid.

### (C) Human LDH5

An enzyme solution was prepared so that the LDH was to be 245 U/mL as a final concentration in a reaction liquid.

From the results of Test Example 3, similar to Test Example 1, also when the porcine LDH1, the human LDH1, and the human LDH5 were used, the enzymatic reactivity of products 1 to 3 was lower than that of the β-NMN compound of Production Example 1.

### Test Example 4

### <Dependency of LDH Activity Value on β-NMN Compound Concentration>

In order to confirm dependency of a LDH activity value on the β-NMN compound concentration, the β-NMN compound produced in Production Example 1 and β-NMN products 1 to 3 the same as those used in Test Example 1 were used for measurement of an enzyme activity of LDH at varied concentrations of the β-NMN in the R1 reagent.

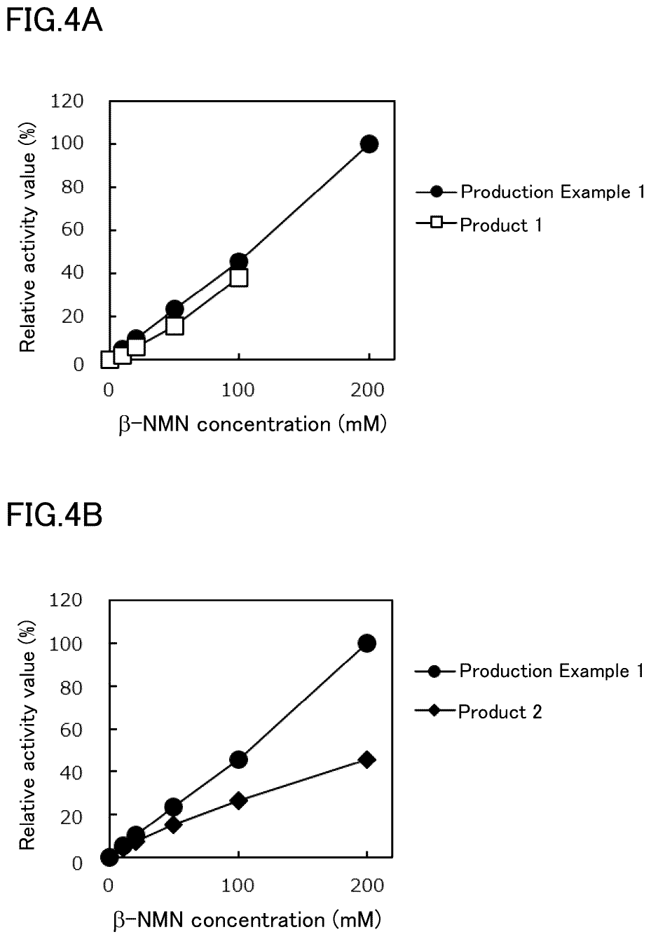

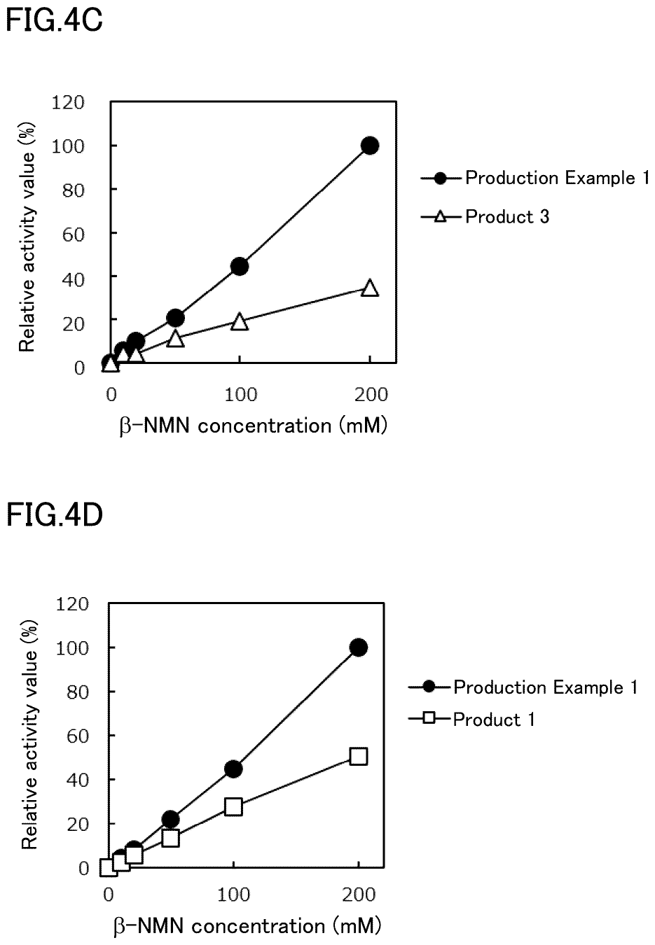

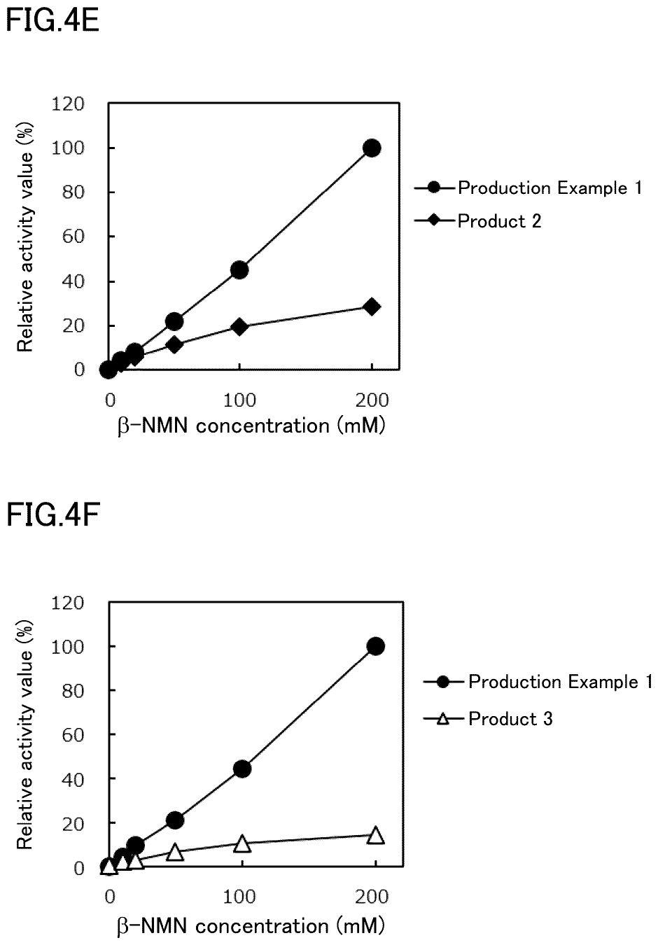

The enzyme activity of LDH was measured in the same manner as in [Evaluation of enzymatic reactivity] of Test Example 1 except that the following R1 reagent and enzyme solution were used, to calculate a value of change in absorbance per minute (ΔmAbs/min). Regarding as 100% the activity value of LDH when the β-NMN compound of Production Example 1 was used at 200 mM, the relative activity values (%) in each NMN at each concentration are presented in FIGS. 4A to 4F (FIGS. 4A to 4C: R-LDH5 and FIGS. 4D and 4F: human LDH1). Note that, the measurement results are average values in the measurement performed three times.

### —R1 Reagent—

### 80 mM Tris-HCl (pH 8.5)

### 0, 10, 20, 50, 100, or 200 mM β-NMN

(The purity of the β-NMN in a measurement sample was assumed to be 100%.
Also, the β-NMN was dissolved in a 100 mM sodium carbonate solution in advance.)

### —Enzyme Solution—

### (I) R-LDH5

An enzyme solution was prepared so that the LDH was to be 245 U/mL as a final concentration in a reaction liquid.

### (II) Human LDH1

An enzyme solution was prepared so that the LDH was to be 45 U/mL as a final concentration in a reaction liquid.

As a result of the measurement, in the case of using the R-LDH5, the product 1 could not be measured for the enzymatic activity at a β-NMN concentration of 200 mM, and the concentration dependency could not be monitored (FIG. 4A). This is likely because of increased inhibition reaction of unknown cause as the β-NMN concentration increased. In the product 2 (FIG. 4B) and the product 3 (FIG. 4C), the difference in the activity value from the β-NMN compound of Production Example 1 became larger as the β-NMN concentration increased.

In the case of using the human LDH1, all of the product 1 (FIG. 4D), the product 2 (FIG. 4E), and the product 3 (FIG. 4F) had larger differences in the activity value from the β-NMN compound of Production Example 1 as the β-NMN concentration increased.

### Test Example 5

### <Effects of NMN on Culture Cells>

In order to evaluate biological activities of the β-NMN compounds of Production Example 1 and the β-NMN product 3, culture cells were cultured in a medium containing the β-NMN compound, to measure change in intracellular NAD.

The culture cells used were HEK293 cells that had been treated to be nonadherent. The medium used was FreeStyle™ 293 Expression Medium (obtained from Thermo Fisher Scientific).

First, the HEK293 cells, which had been treated to be nonadherent, were disseminated in the medium so as to have a concentration of 1×10⁶ cells/mL. The resulting medium was dispensed into six 125 mL-flasks by 30 mL each. The flasks were supplemented with the β-NMN compounds of Production Example 1 and the β-NMN product 3 so as to have a final concentration of 0 or 0.1 mM. Without exchange of the medium, the cells were shake-cultured for seven days in a CO₂ incubator at 37° C.

After shake-culturing for seven days, the cells and the culture supernatant were recovered for various analyses. First, in order to calculate cytotoxicity rates, as detection of an escape enzyme, L-type Wako ASTJ2 (obtained from FUJIFILM Wako Pure Chemical Corporation) was used for intra- and extracellular aspartate aminotransferase (AST) activities, followed by measurement using a 7180 type Hitachi automatic analyzer (obtained from Hitachi High-Tech Corporation) according to the method specified for the reagents. Also, in order to measure the intracellular NAD content, NAD was assayed using a NAD/NADH measuring kit (obtained from DOJINDO LABORATORIES). Specifically, the recovered cells were weighed and were extracted with Extraction buffer attached to a kit, and the resulting extract was measured for NAD. The intracellular NAD content was calculated based on a calibration curve obtained by measuring NAD solutions of various concentrations.

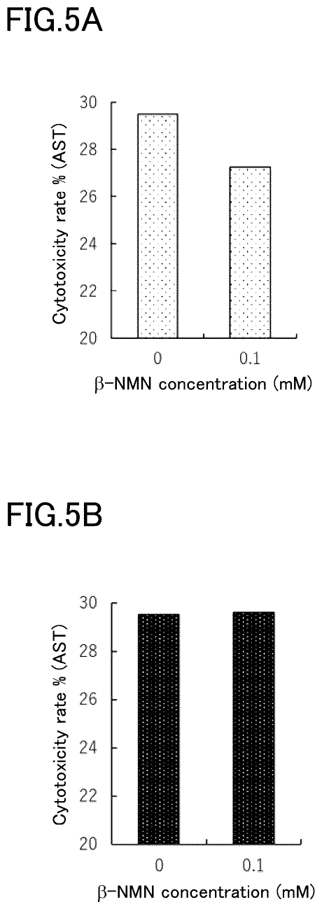

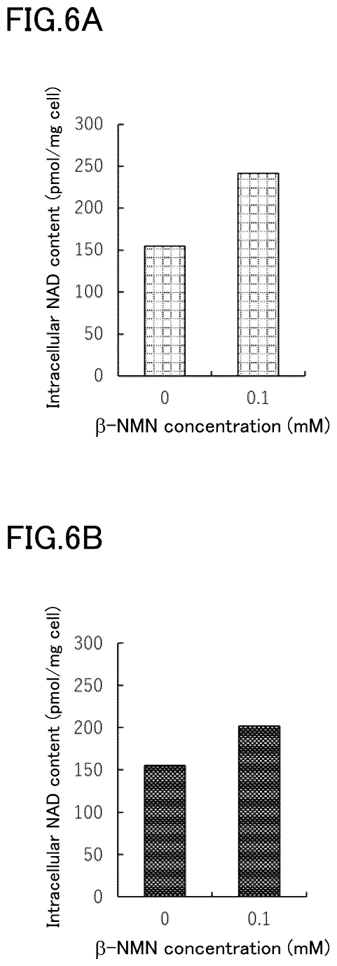

FIG. 5A illustrates cytotoxicity rates determined based on the intra- and extracellular AST activities when the β-NMN compound of Production Example 1 was used, and FIG. 5B illustrates cytotoxicity rates determined based on the intra- and extracellular AST activities when the β-NMN compound of the β-NMN product 3 was used. Also, FIG. 6A illustrates the intracellular NAD content when the β-NMN compound of Production Example 1 was used, and FIG. 6B illustrates the intracellular NAD content when the β-NMN compound of the β-NMN product 3 was used. As a result of the measurement, the cytotoxicity rate due to depletion of nutrients when cultured for seven days without exchanging the medium was lower when the β-NMN compound of Production Example 1 was added than when the β-NMN compound of the β-NMN product 3 was added. Also, the intercellular NAD content was higher when the β-NMN compound of Production Example 1 was added than when the β-NMN compound of the β-NMN product 3 was added.

1. A compound, comprising:
β-nicotinamide mononucleotide or a pharmacologically acceptable salt thereof, wherein a purity of the compound as measured through HPLC is 95% or higher, and a reactivity of the compound with lactate dehydrogenase is 30 units or higher.

2. The compound according to claim 1, wherein the purity of the compound as measured through HPLC is 98% or higher, and the reactivity of the compound with lactate dehydrogenase is 33 units or higher.

3. The compound according to claim 1, wherein the compound is in crystalline form.

4. The compound according to claim 1, wherein the lactate dehydrogenase is lactate dehydrogenase derived from mammalian skeletal muscle.

5. The compound according to claim 4, wherein the lactate dehydrogenase includes an amino acid sequence of SEQ ID NO: 1.

6. The compound according to claim 1, wherein the compound is substantially free from nicotinamide dinucleotide.

7. A method of evaluating a quality of a compound including β-nicotinamide mononucleotide or a pharmacologically acceptable salt thereof, the method comprising:
evaluating the quality of the compound including the β-nicotinamide mononucleotide or the pharmacologically acceptable salt thereof based on an indication that a purity of the compound as measured through HPLC is 95% or higher, and a reactivity of the compound with lactate dehydrogenase is 30 units or higher.

8. A method of determining an enzymatic reactivity of a compound including β-nicotinamide mononucleotide or a pharmacologically acceptable salt thereof, the method comprising:
determining a reactivity, with lactate dehydrogenase, of the compound including the β-nicotinamide mononucleotide or the pharmacologically acceptable salt thereof based on an indication that a purity of the compound as measured through HPLC is 95% or higher, and the reactivity of the compound with the lactate dehydrogenase is 30 units or higher.
